(19) United States
(12) Patent Application Publication
(10) Pub. No.: US 2013/0177616 A1
(43) Pub. Date: Jul. 11, 2013

(54) NANOSTRUCTURED SUN PROTECTION AGENT AND PROCESS

(71) Applicant: Instituto de Pesquisas Tecnologicas do Estado de Sao Paulo S.A.-IPT, Sao Paulo (BR)

(72) Inventors: Adriano Marim de Olivera, Sao Paulo (BR); Kleber Lanigra Guimaraes, Sao Paulo (BR); Natalia Neto Pereira Cerize, Sao Paulo (BR)

(73) Assignee: Instituto de Pesquisas Tecnologicas do Estado de Sao Paulo S.A.-IPT, Sao Paulo (BR)

(21) Appl. No.: 13/622,345

(22) Filed: Sep. 18, 2012

Related U.S. Application Data
(63) Continuation of application No. PCT/BR2011/000076, filed on Mar. 21, 2011.

(30) Foreign Application Priority Data
Mar. 19, 2010 (BR) P1 1000919-1

(51) Int. Cl. A61K 8/11; A61Q 17/04

(52) U.S. Cl. CPC A61K 8/11; A61Q 17/04
USPC 424/401; 424/59

(57) ABSTRACT

A system comprising core-shell-type nanoparticles is described, the shell consisting of oxide nanoparticles and the core consisting of polymers and solar radiation protection chemicals, said system providing broad-spectrum solar protection, ranging from UVA to UVB, due to the chemical composition thereof which comprises physical protection agents, oxide nanoparticles and nanoencapsulated chemical protection agents within the polymeric matrix. Due to the size scale, composition and morphology of said prepared nanoparticles, these can be used in cosmetic formulations, in preparing sunscreens, or in any other formulation mainly intended to provide solar radiation protection.

### RELEVANT FIELD

The invention relates to the sector of cosmetics and pharmaceutical (medicinal) preparations of active ingredients typified by special, nanostructured physical forms (nano-particulates) of the core-shell type, containing oxide particles on their surface and active chemical agents inside, which confer protection against UVA and UVB-type radiation (light). Due to their size scale, composition and morphology, these nanoparticles may be applied in cosmetic formulations for the preparation of sunscreens, or in any other formulation with the primary purpose of protection from solar radiation.

### OBJECT OF THE INVENTION

The object of the invention is to present a nanostructured sun protection agent that confers a UVA and UVB type solar protection factor, integrated into a single nano-particulated system and its production process.

### BRIEF SUMMARY OF THE INVENTION

The solar protection agent is obtained by polymerization of ethylene monomers in an emulsion in the presence of solid, colloidal particles, and by at least one chemical light-absorbing agent, forming core-shell type nanoparticles, where the shell is composed of nanoparticles of oxides and the core is composed of polymers and at least one chemical agent to protect against solar radiation, which confer solar protection across a wide spectral band, ranging from UVA to UVB. The chemical light-absorbing agents used in this nanoencapsulation may be any already known in the prior art that are normally used in the preparation of sunscreens.

In order for this solar protection agent to provide this type of protection from light (UVA and UVB), it uses a process for preparation of the nanoparticles that consists of the emulsion polymerization of acrylate-type ethylenic monomers, using particles of colloidal oxide as a stabilizer, and by means of this polymerization, the encapsulation of the chemical light-absorbing agent occurs in situ.

In this nanostructured system, the colloidal oxide particles used as a stabilizer during polymerization are anchored to the surface of the nanoparticles, thus providing a physical barrier to the passage of light, primarily wavelengths in the UVB range, and the chemical agent that was nanoencapsulated, and is within the nanoparticles, provides protection primarily in the UVA region. In this manner, this nanostructured sun protection agent can provide physical and chemical protection at the same time for a sun protection formulation, thus avoiding risks of irritation and allergic reaction that are common to the hybrid protection systems normally used.

Other benefits associated with this sun protection agent consist of the possibility of working on a nanometric scale, which provides greater covering capacity during the dermal application of the product, i.e., greater surface area of the nanoparticles, in addition to the fact that these particles are on the order of size of the radiation that is to be diffused. The association of physical and chemical protective effects provided to the chemical agent for absorption of UV rays by the nanoencapsulation process is also noteworthy.

Thus, several of these chemical agents may be easily degraded when exposed to UV radiation, which may lead to a deficiency in the solar filter protection factor, while the photodegradation occurs primarily in the UVA radiation range. In addition, these chemical agents cannot dissipate the energy of the excited state as efficiently as melanin, and, therefore, penetration of these ingredients into the deepest layers of the skin may result in an increase in the production of free radicals and reactive oxygen species (ROS). Furthermore, another benefit of the proposed system is the protection of the user against possible toxic chemical compounds formed from photodegradation of the chemical agent, which in some cases may be more harmful to the skin than the light itself (Wright et al, 2001).

Lastly, we have the benefit of the ability to work with greater concentrations of the chemical agent in the solar protection formulations, without this causing skin irritability or irritation, given that it is nanoencapsulated in a polymer matrix and coated with a layer of inorganic particles, thus the nanostructured system will avoid the chemical agent having the same toxicity effect compared to situations in which it is free in the formulation.

### PRIOR ART

The solar spectrum that reaches the earth's surface comprises predominantly ultraviolet radiation (100-400 nm), visible radiation (400-800 nm) and infrared radiation (above 800 nm) (Wolf et al, 2001).

The human body perceives the presence of these radiations in the solar spectrum in varying manners. Infrared (IV) radiation is perceived in the form of heat, visual (Vis) radiation through the various colors detected by the optical system, and ultraviolet (UV) radiation through photochemical reactions. Such reactions may stimulate the production of melanin, the manifestation of which is visible in the form of tanning of the skin, or may range from causing simple inflammation to serious burns. The possibility also exists of genetic mutations and abnormal cell behavior (Wolf et al, 2001).

Research has shown that UV radiation damages the DNA and genetic material, oxidizes lipids producing dangerous free radicals, cause skin inflammation, breaks down cellular communication, modifies the expression of genes in response to stress and weakens the immune response of the skin. The use of sunscreens is intended to reduce the quantity of UV radiation to be absorbed by the human skin, serving as a protective barrier (Angeli, 2007).

Energy from solar radiation increases with the reduction of its wavelength, thus UV radiation is that with the shortest wavelength, and as a result, it contains the most energy, i.e., the greatest propensity to cause photochemical reactions. Another important consideration relates to the capacity of this radiation to permeate the structure of the skin, given that low-energy UV radiation (320 to 400 nm) penetrates deeper into the skin and, when reaching the dermis, is responsible for photo-aging, possibly causing skin cancer. This radiation, known as UVA radiation, has a constant intensity and varies little throughout the day and throughout the year (Gawkrodger, 2002; Schulz et al, 2002).

UVB radiation penetrates the surface of the skin, given that it has high energy, and frequently causes sunburn. It also causes the skin to tan, being responsible for the transformation of epidermal ergosterol into Vitamin D, and causes early aging of cells. Frequent and intense exposure to this radiation may cause damage to the DNA, in addition to suppressing the skin's immune response. In this manner, in addition to increasing the risk of fatal mutations, manifested in the form of skin cancer, its activity reduces the chance of a malignant cell being recognized and destroyed by the body (Angeli, 2007; Gawkrodger, 2002; Schulz et al, 2002).

The endogenous mechanism for protection against UV radiation may be associated with the presence of melanin (a pigment produced by melanocytes, basal epidermal cells), which, among other functions, gives color to the skin. Moderate exposure to sunlight results in an increase in the production of melanin and the consequent tanning. This pigment acts to absorb UV radiation and to dissipate energy in the form of heat, preventing cutaneous damage to the skin tissue. UVA radiation leads to oxidation of the melanin and the release of pigments produced previously, contained in the mealnocytes, just as UVB causes temporary tanning and stimulates the production of more melanin. The chemical properties of melanin make it an excellent photoprotector that is more efficient that conventionally used sunscreens. The penetration of these compounds into the deepest layers of the skin can increase the quantity of free radicals and of reactive oxygen species (ROSs) (Angeli, 2007).

In this context, sunscreens have been widely used, in order to reduce damage caused by solar radiation.

In addition to absorbing incident ultraviolet radiation, a sun protection product must be stable on the human skin and in heat, and be photostable under sunlight to allow protection lasting several hours, preventing contact with degradation products. At the same time, sunscreens must also not be irritants, sensitizing agents or phototoxic substances. They must coat and protect the surface of the skin, but they must not penetrate it, in order to not cause systemic exposure to these substances. Sunscreens must not be toxic, since traces of them are absorbed through the skin or are ingested after application to the lips. Another important feature of sunscreens is their compatibility with cosmetic formulations (Fiori J et al, 2007).

There are two classes of sunscreens: organic and inorganic, classified routinely and respectively as chemical effect filters (chemical filters) and physical effect filters (physical filters). Generally, the organic compounds protect the skin by absorbing radiation and the inorganic compounds protect by reflecting radiation. On the market, there are currently organic filters that in addition to absorbing UV radiation also reflect it. It is noted that the phenomena of reflection and diffusion depend, among other factors, on the size of particles of the inorganic filter, and on the fact of whether it is an organic or inorganic chemical compound (Flori J et al, 2007).

Inorganic sunscreens are used to prevent damage caused by UV radiation, acting primarily through the reflection and diffusion of light. These are considered a safer and more effective method of protecting the skin, since they present a low potential for irritation, including sunscreens recommended in the preparation of photoprotectors for use on children and persons with sensitive skin. It must be noted that inorganic filters, of adequate size, in addition to absorbing UV light, also diffuse it (Flori J et al, 2007).

Zinc oxide and titanium dioxide are the primary compounds used as inorganic filters, and when they are included in formulations they are suspended, with the size of particulates being of prime importance not only in the efficacy of the sunscreen but also in the physical (cosmetic) appearance of the product. A negative point in the use of this type of sunscreen is the tendency to leave a white film on the skin, which may be aesthetically undesirable (Angeli, 2007).

The maximum diffusion of light occurs in the presence of particles with a diameter approximately equal to the wavelength of the incident light. In order to not cause the formation of a white film on the skin, the size of particles cannot be on the same order of the size of the wavelength of the visible radiation range, so the particles must be less than 400 nm (Angeli, 2007).

Some interactions may occur that are not very favorable, associated with the use of inorganic sunscreens. The microfine pigments require being adequately dispersed in the vehicle, normally an emulsion, in order to be effective, and poor dispersal will reduce the performance of the product. Microfine pigments also must be kept in suspension, so that the particles do not agglomerate, since the final performance of the product will decrease if flocculation/agglomeration (coalescence) and the formation of major aggregates occurs during storage (Flori J, et al, 2007).

At the same time, organic filters are formed by organic molecules that are capable of absorbing UV (high-energy) radiation and transform it into radiation with lower energy levels and that are harmless to the body. These molecules are essentially aromatic compounds with carboxylic groups. In general, they present a donor group of electrons, which when they absorb UV radiation are excited by the empty orbit of lower energy and, upon returning to their basic state, the excess energy is released in the form of heat. It is noted that the efficacy of these compounds is dependent on their capacity to absorb radiant energy, which is proportional to their concentration, absorption range and, primarily, the wavelength at which maximum absorption occurs. This being the case, several compounds have greater efficacy in the UVA range, while others have a maximum absorption peak and are more efficient in the UVB range (Flori J, et al, 2007).

Since sunscreens absorb only a portion of the ultraviolet range (UVA and UVB), in order to have complete protection, combinations of these filters are used. On the other hand, the combination of different types of filters may cause a high degree of irritability when applied to the skin.

Some inventions have already been patented using nanoparticles of sunscreens, the majority of which relate to the development and application of physical filters in the form of nanoparticles; however, none of them demonstrates the ability to bring together two sun protection mechanisms (physical and chemical) in the same nanoparticle, which can give the sunscreen formulation greater versatility, economy, ease of formulation and fewer problems with physical and/or chemical stability, since this is basically the novelty and the inventive step of the “NANOSTRUCTURED SOLAR PROTECTION AGENT AND PROCESS,” which is the object of this patent.

Patent WO/2003/072077 “A Substantially Visibly Transparent Topical Physical Sunscreen Formulation” describes a system containing physical filter nanoparticles (ZnO), with broad protection (UVA and UVB), coated with a metallic oxide or hydroxide, dispersed with a surfactant.

Another composition, in US 2009117384-A1 “Metal oxide nanoparticle containing composition for UV-blocking composition used in sunscreen composition, has regular polyhedral nanocavities which are isolated from surface of nanoparticle,” describes only one physical form of protection against UV rays using nano-particulated TiO₂ containing polyhedral cavities on the surface. This system may be used in making UV blocking formulas, in cosmetic application (sunscreen); industrial coating, and also in solar energy conversion systems and lithium batteries. Nanoparticles with cavities have greater UV absorption (at wavelengths below 360 nm) when compared to those without cavities. Through treatment of powdered oxide with an alkaline solution (in an autoclave at 150-190° C.), washing with an acid solution and heating to 550-750° C. in an oxygen and ammonia atmosphere, nanoparticles with polyhedral cavities are formed.

Document US 2005/0208087 (Modified Oxidic Nano-particle with Hydrophobic Inclusions, Method for the Production and Use of said Particle) also describes the production of nanoparticles of a physical filter, modified with hydrophobic inclusions comprising halogenated molecules. The primary uses are related to toner, cosmetic sunscreens, insecticides or biomolecule markers.

Invention U.S. Pat. No. 5,955,091 (Photobluing/whitening-resistant cosmetic/dermatological compositions comprising TiO₂ pigments and deformable hollow particulates) deals with a cosmetic composition, or sun screen, or dermatological composition, for topical use, indicated to improve photoprotection of the human skin and/or the hair, including at least one nano-pigment (TiO₂) and an effective quantity of hollow deformable particles (microspheres) in a size range of between 1 and 250 micrometers, comprising a vinylidene chloride, acrylonitrile and methacrylate copolymer.

U.S. Pat. No. 7,344,591 (Stabilized titanium dioxide nanoparticle suspension, e.g. for personal care application such as sunscreen, comprises dispersing agent containing organic molecules that have [a] functional group, e.g. hydroxyl and/or carboxyl) describes a TiO₂ suspension, solvent-free nanoparticulate, stabilized in a type of alcohol.

The work that presents several aspects similar to the present invention is GB 2453195 (Aqueous nano-particulate dispersion comprising a UV absorber/polymer mixture for pharmaceutical/cosmetic applications) that is characterized by the presence of a carrier polymer (prepared by radical polymerization in a dispersed medium) with ethylenically unsaturated units and a lyophilic solar UV filter at concentrations of approximately 2:1 weight ratio of polymer to filter. This carrier system (described as an aqueous polymer dispersion) is incorporated into a cosmetic composition exhibiting solar protection effects and a sensation that is pleasing to the skin. One aspect of this suspension is that the particle size is below 1000 nm and another is that it uses cationic or non-ionic surfactants in the process of obtaining it. Similarly, the present invention “NANO-STRUCTURED SOLAR PROTECTION AGENT AND PROCESS” presents a polymer carrier system containing a solar filter; however, it is emphasized that the constitution and structure, process for obtaining it and the benefit obtained in application are novelties with substantive differences relative to that work.

The nano-structured agent cited in “NANO-STRUCTURED SOLAR PROTECTION AGENT AND PROCESS” comprises core-shell nanoparticles, where the shell is comprises nanoparticles of oxide and the core comprises polymers and a chemical agent for protection against solar radiation, which confers solar protection in a wide spectral range, varying from UVA to UVB. In the case of Patent GB 2453195, the inventors did not use oxide particles to stabilize colloidal dispersions and that also confer protection against solar radiation, using only conventional emulsifiers, which may bring about limitations in relation to topical administration and effects of dermal irritability. Due to their chemical composition, which contain physical protection agents, oxide nanoparticles and chemical protection agents nano-encapsulated in the polymer matrix, as well as their size scale, composition and morphology, the nanoparticles prepared here may be applied in cosmetic formulations for the preparation of sunscreens or in any other formulation that has the primary objective of protecting against solar radiation.

The presentation of a nano-structured solar protection agent that confers a UVA and UVB solar protection factor integrated into the same nano-particulate system containing physical and chemical protectors is a novelty and inventive step in the concept of a sunscreen in regard to the mechanism of action of these components.

### DESCRIPTION OF FIGURES

FIG. 1A presents the turbidimetric curves obtained from the polymer dispersion synthetized under the experimental conditions of Example 1.

FIG. 1B presents the wavelength (nm) spectrum of transmittance X of a sample of the solar protection agent, at a concentration of 0.005% by weight, obtained in accordance with the experimental conditions of Example 1.

FIG. 2A presents the turbidimetric curves obtained from the polymer dispersion synthesized under the experimental conditions of Example 2.

FIG. 2B presents the wavelength (nm) spectrum of transmittance X of a sample of the solar protection agent, at a concentration of 0.005% by weight, obtained in accordance with the experimental conditions of Example 2.

FIGS. 2C and 2D present microscopy images of the product prepared in accordance with Example 2.

FIG. 3A presents the turbidimetric curve obtained from the polymer dispersion synthesized under the experimental conditions of Example 3, showing a small phase separation, indicated by the reduction of intensity of “backscattering” after 3 days of analysis.

FIG. 3B presents the wavelength (nm) spectrum of transmittance X of a sample of the solar protection agent, at a concentration of 0.005% by weight, obtained in accordance with the experimental conditions of Example 3.

FIG. 4A presents the turbidimetric curve obtained from the polymer dispersion synthesized under the experimental conditions of Example 4, showing a small phase separation, indicated by the reduction of intensity of “backscattering” after 3 days of analysis.

FIG. 4B presents the wavelength (nm) spectrum of transmittance X of a sample of the solar protection agent, at a concentration of 0.005% by weight, obtained in accordance with the experimental conditions of Example 4.

FIG. 5A presents the turbidimetric curve obtained from the polymer dispersion synthesized under the experimental conditions of Example 5, showing a small phase separation, indicated by the reduction of intensity of “backscattering” after 3 days of analysis.

FIG. 5B presents the wavelength (nm) spectrum of transmittance X of a sample of the solar protection agent, at a concentration of 0.005% by weight, obtained in accordance with the experimental conditions of Example 5.

FIG. 6 presents a graphic superimposing the wavelength (nm) curves of transmittance X under UV spectroscopy of the nano-structured solar protection agent, at a concentration of 0.005% by weight, with the plotted curve corresponding to a nanoparticle without 3-benzophenone; the dotted curve corresponding to Example 1; the dotted curve is plotted corresponding to Example 2; and the solid line curve corresponds to Example 3.

### DETAILS OF THE INVENTION

The “NANOSTRUCTURED SOLAR PROTECTION AGENT AND PROCESS” presented here is a product with the combination of two solar protection mechanisms (physical and chemical) in the same nanoparticle that has a shell-core type morphology, where the shell is formed by nanoparticles of oxides, such as silica, titanium oxide, zinc oxide, and the core is formed by a polymer, which may be of the acrylate, methacrylate, vinyl, styrenic, acrylamide, methacrylamide types, preferably acrylates and methacrylates, and a chemical agent for protection against any solar radiation corresponding to the prior art.

The synthesis of this solar protection agent is carried out using the usual polymerization technique, which may be conducted in a conventional polymerization reactor equipped with a mechanical agitator, reflux condenser and water circulation housing. The colloidal dispersion of the oxide (in a proportion by weight of from 0.01 to 10%, preferably 1%, in relation to the final product, with appropriate granulometry in relation to the granulometry of the product) is prepared directly in the reactor and heated to the reaction temperature. Then, the charge of monomers is added, comprising a mixture of hydrophobic monomer (in a proportion of from 1 to 70% by weight, preferably 20%, in relation to the final product) which will produce the polymer in question together with the chemical agent for solar protection (in a proportion of from 0.0005 to 30%, preferably 4%, in relation to the final product), which is previously solubilized in the monomers. After the addition of the monomers and a slight homogenization of the reaction medium (from 10 to 1000 rpm, preferably 200 rpm), and after adding the charge of initiator that will be responsible for initiating the polymerization reaction. The polymerization reaction is conducted for varying times, which can range from 1 up to 24 hours of reaction and at temperatures ranging from 50 to 100° C., depending on the materials used. At the end of the reaction, the polymer dispersion can be used as is, without any post-processing stage, or recovered in the form of a powder by conventional particle drying methods, such as lyophilization or spray drying.

In order to confirm the formation of the solar protection agent, nanometric dispersions obtained in an aqueous medium can be characterized by various techniques, such as: laser diffraction, zeta-potential, turbidimetry, gravimetry, potentiometry, tensiometry and electronic microscopy, high-performance liquid chromatography and ultraviolet spectroscopy.

### EXAMPLES

### Example 1

### Obtaining a Solar Protection Agent Containing 3-Benzophenone (3-BZ) Nanoencapsulated by Poly(Methyl Methacrylate).

In a shielded glass reactor with a bottom outlet with a volume of 200 mL, 80 g of a colloidal silica suspension with a concentration by weight of 1% was added. After adjusting the temperature to 70° C. 20 g of methyl methacrylate and 1 g of 3-benzophenone were added, while agitating at a rate of 150 rpm and the reaction was started by adding 0.2 g of potassium persulphate dissolved in 10 g of deionized water. The reaction was conducted at this temperature for 4 hours and after that time the polymer dispersion obtained was described.

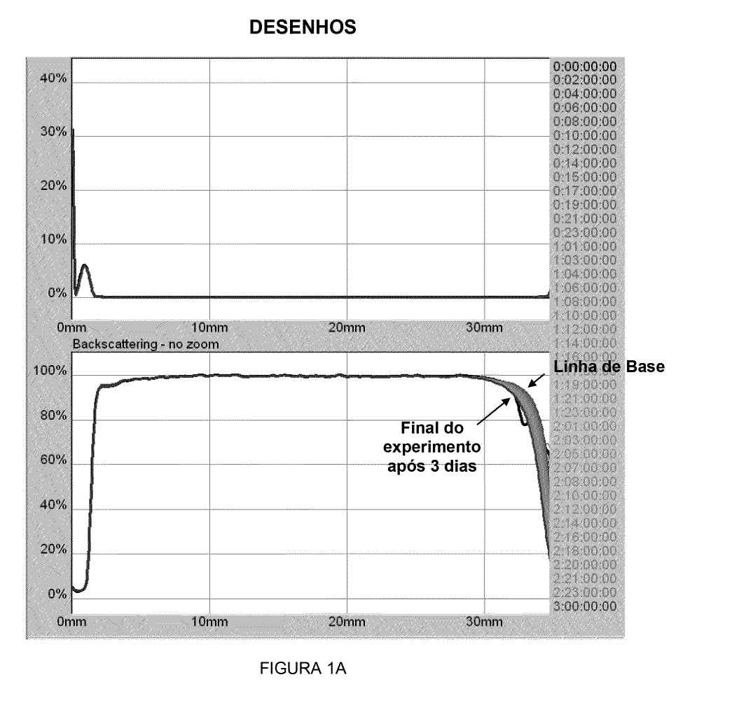

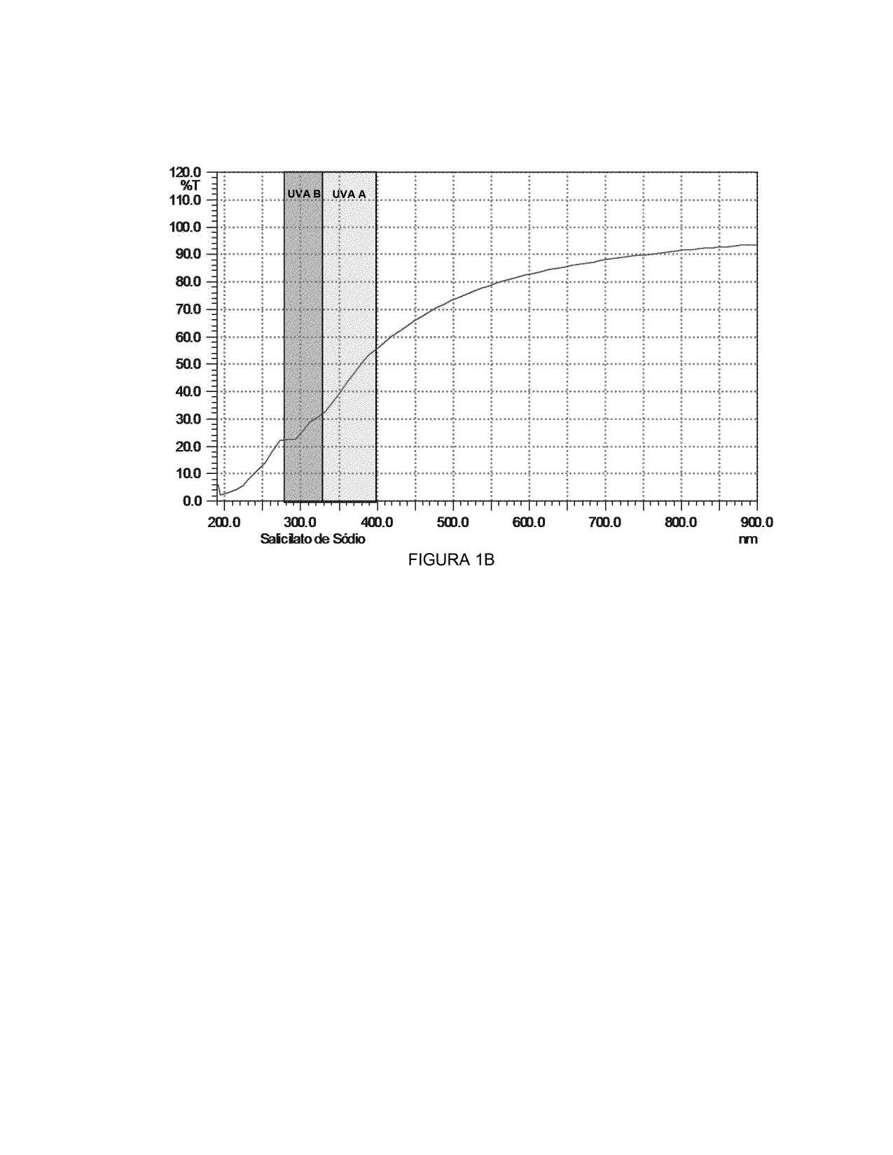

The results obtained in this example are shown in Table 1. FIG. 1A presents the turbidimetric curves obtained with the polymer dispersion synthesized under the experimental conditions of this example. This result demonstrates that for this colloidal dispersion sample a small phase separation occurred, indicated by the reduction in intensity of the backscattering after 3 days of analysis. However, this separation is reversible, since gentle agitation of the flask is sufficient to resuspend the particles. FIG. 1B presents a transmittance curve of a sample diluted to a concentration of 0.005% by weight. This result shows the capacity of the nanoparticles to absorb light, primarily in wavelengths shorter than 400 NM.

TABLE 1
Results obtained with the product produced using
the experimental conditions of Example 1.

| Solids | | 3-BZ | Zeta | | | | |
|---|---|---|---|---|---|---|---|
| Content | | Content | Potential | Viscosity | Particule Size (nm) | | |
| (%) | pH | (%) | (mV) | (cPs) | D₁₀ | D₅₀ | D₉₀ |
| 15.3 | 3.85 | 84 | −56.1 | 6.00 | 227 | 286 | 359 |

### Example 2

### Obtaining a Solar Protection Agent Containing 3-Benzophenone Nanoencapsulated in Poly(Methyl Methacrylate)

In a shielded glass reactor with a bottom outlet with a volume of 200 mL, 80 g of a colloidal silica suspension was added, with a concentration by weight of 1%. After adjusting the temperature to 70° C., 20 g of methyl methacrylate and 2 g of 3-benzophenone were added, while agitating at a rate of 150 rpm and the reaction was initiated by adding 0.2 g of potassium persulphate dissolved in 10 g of deionized water. The reaction was conducted at this temperature for 4 hours and after this period of time the polymeric dispersion obtained was described. The results obtained for this example are presented in Table 2.

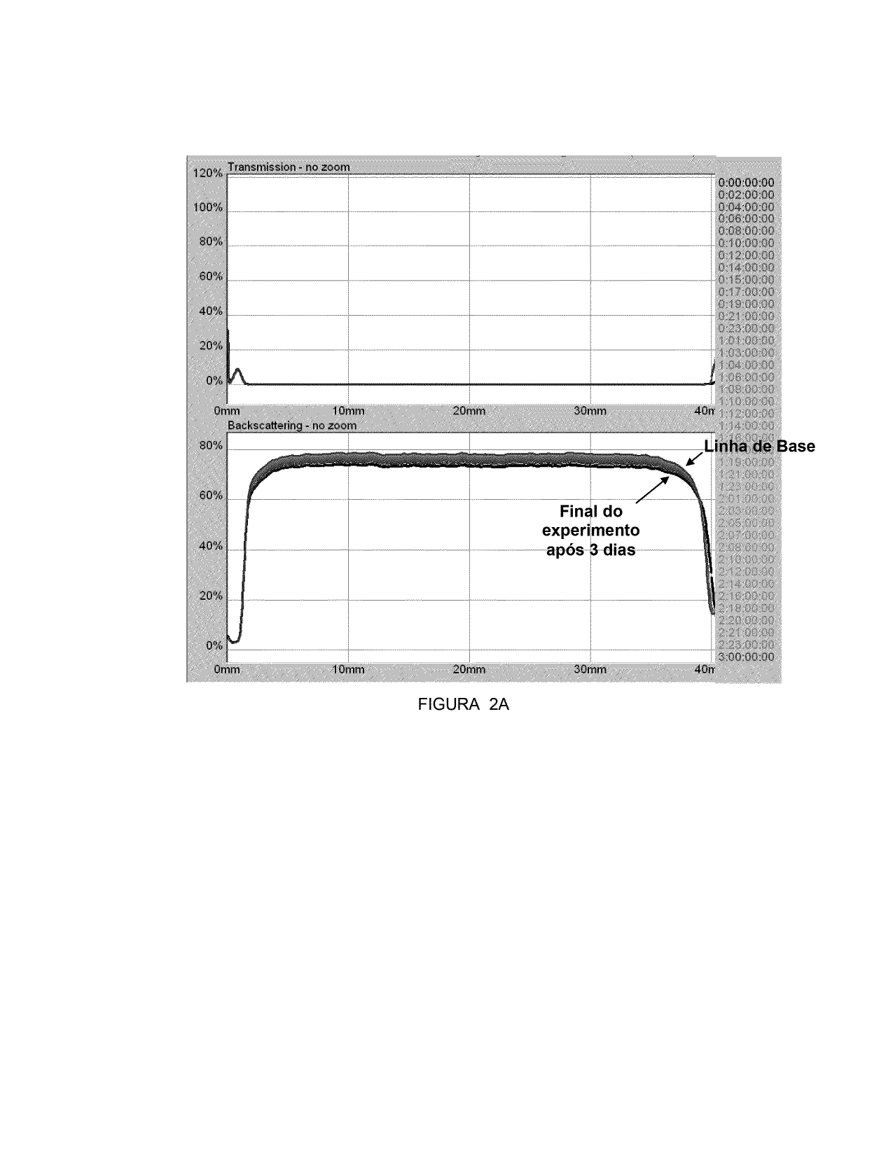

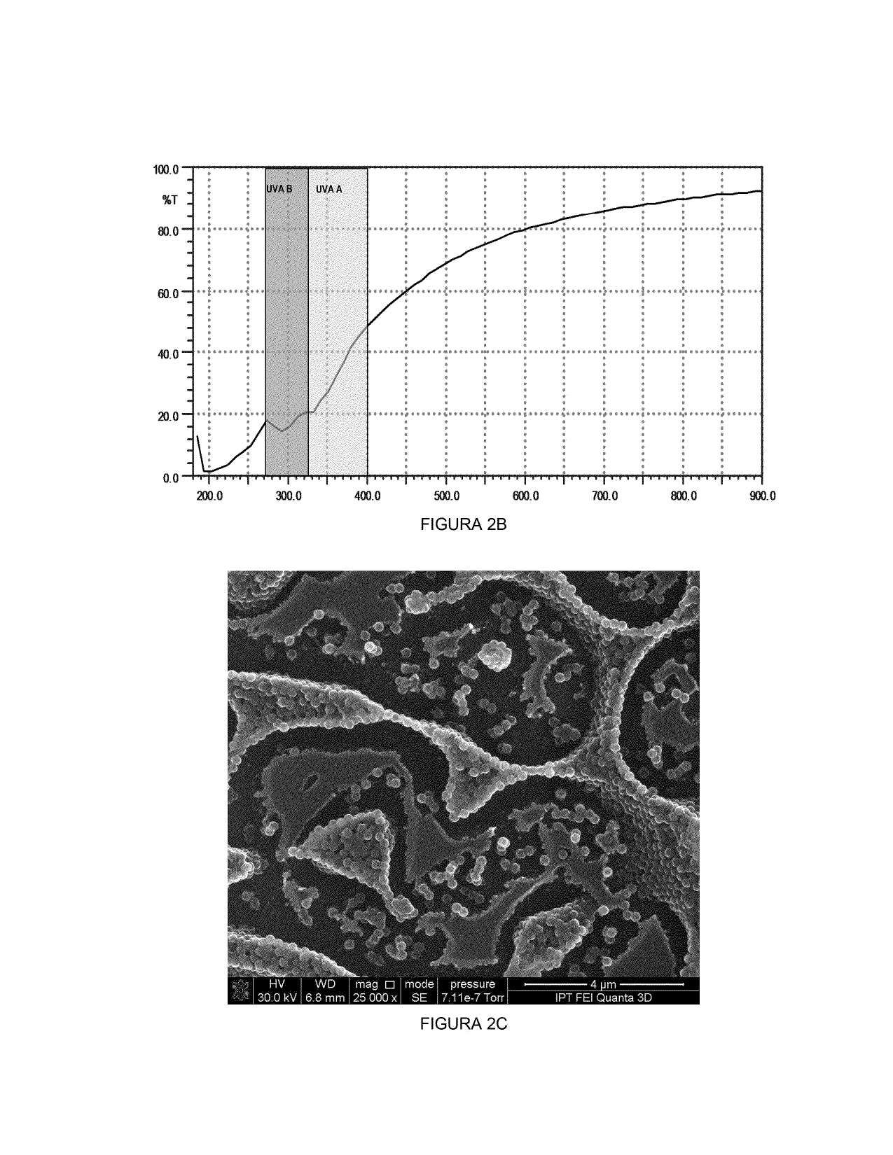

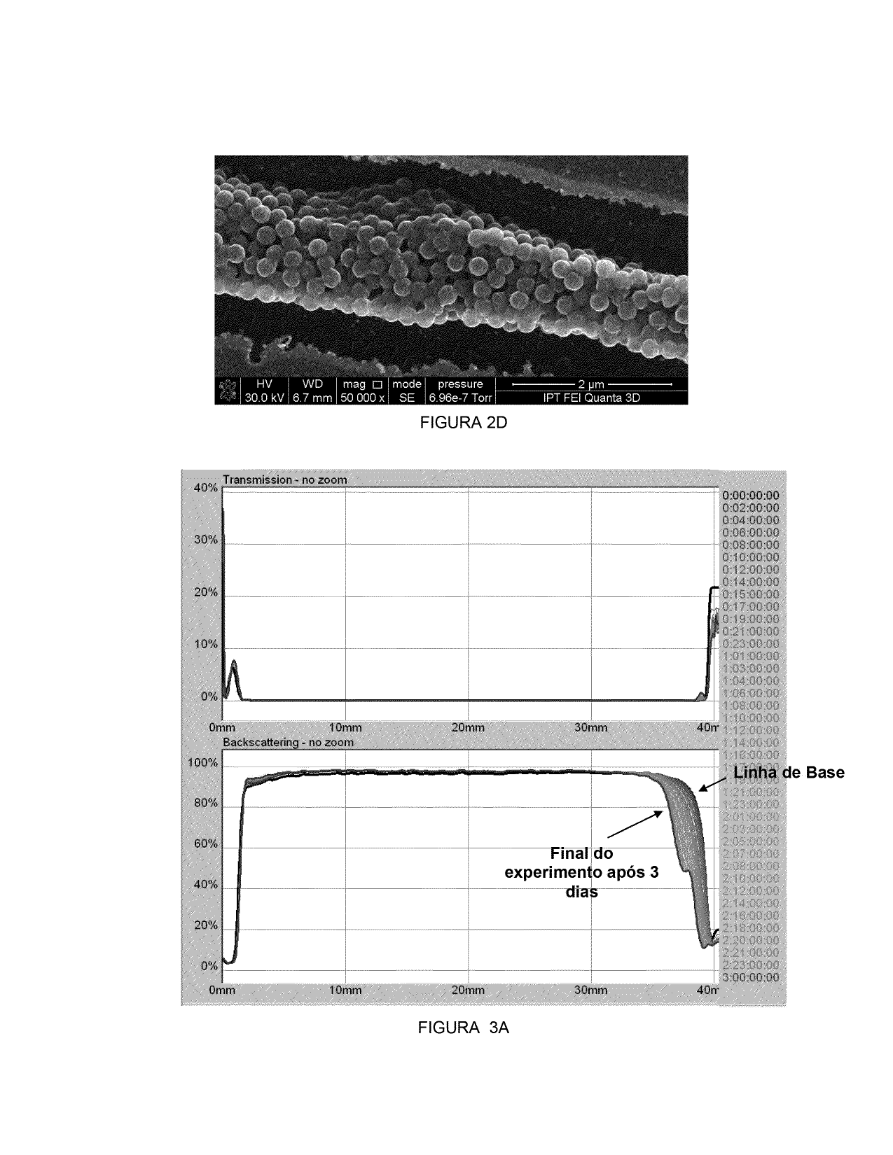

FIG. 2A presents the turbidimetric curves obtained with the polymeric dispersion synthesized under the experimental conditions of this example. FIG. 2B shows the dilution transmittance curve of a sample of nanoparticles up to a concentration of 0.005% by weight, and it is possible to observe how the passage of light is blocked at wavelengths below 400 nm. FIGS. 2C and 2D show electronic scanning microscopy images of the field emission type (MEV-FEG) of the product obtained in Example 2.

TABLE 2
Results obtained with the product produced using
the experimental conditions of Example 2.

| Solids | | 3-BZ | Zeta | | | | |
|---|---|---|---|---|---|---|---|
| Content | | Content | Potential | Viscosity | Particule Size (nm) | | |
| (%) | pH | (%) | (mV) | (cPs) | D₁₀ | D₅₀ | D₉₀ |
| 19.4 | 3.60 | 97 | −57.6 | 3.25 | 279 | 368 | 606 |

### Example 3

### Obtaining a Solar Protection Agent Containing 3-Benzophenone Nanoencapsulated in Poly(Methyl Methacrylate)

In a shielded glass reactor with a bottom outlet with a volume of 200 mL, 80 g of a colloidal silica suspension was added, with a concentration by weight of 1%. After adjusting the temperature to 70° C., 20 g of methyl methacrylate and 4 g of 3-benzophenone were added, while agitating at a rate of 150 rpm and the reaction was initiated by adding 0.2 g of potassium persulphate dissolved in 10 g of deionized water. The reaction was conducted at this temperature for 4 hours and after this period of time the polymeric dispersion obtained was described. The results obtained for this example are presented in Table 3.

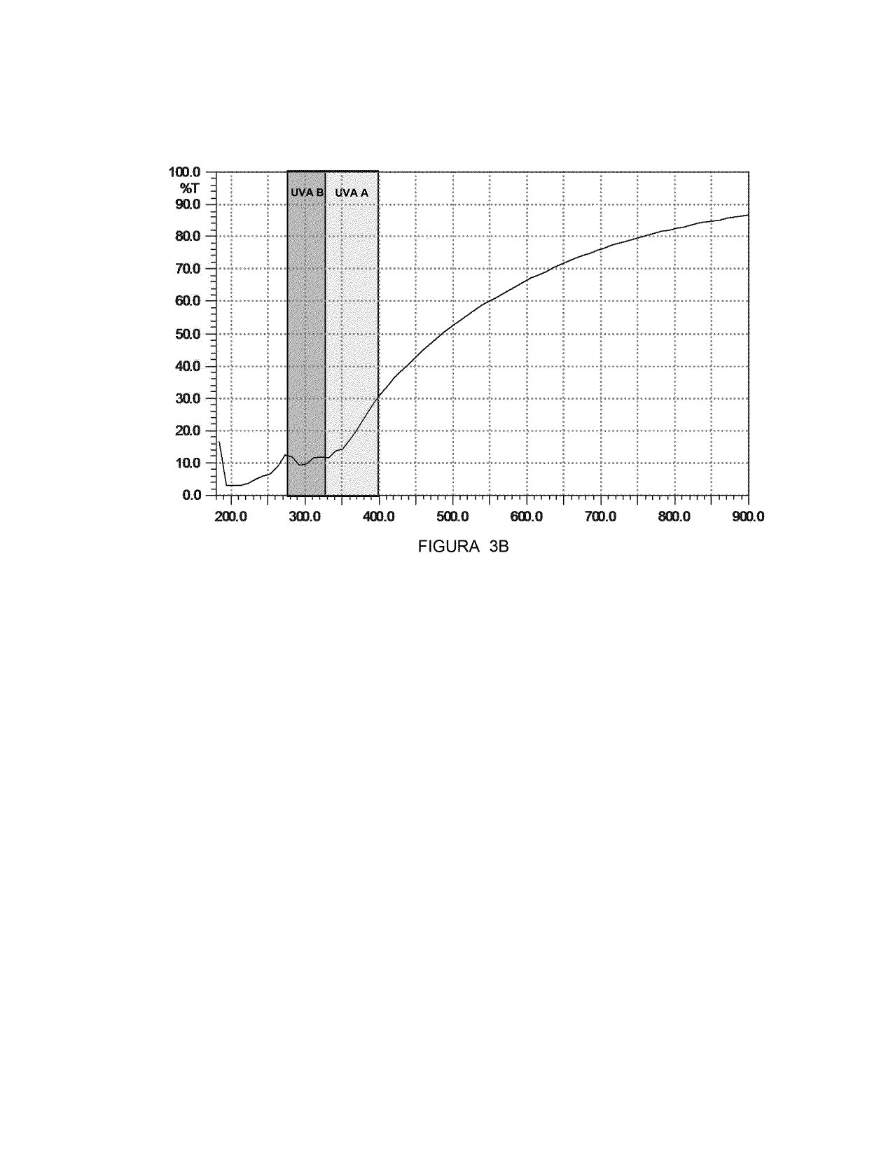

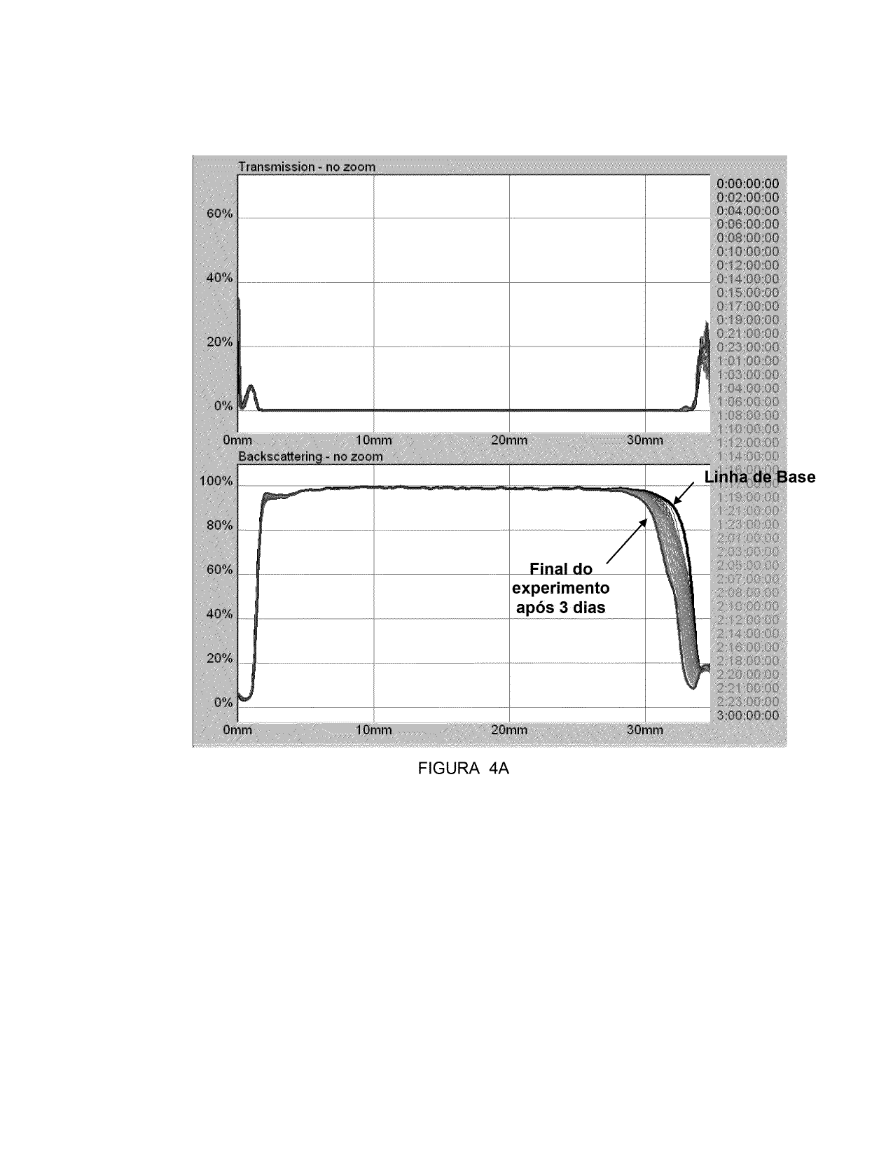

FIG. 3A presents the turbidimetric curves obtained with the polymer dispersion synthesized under the experimental conditions of this example.

This result also showed a small phase separation, indicated by a reduction in the backscattering intensity after 3 days of analysis. In FIG. 3B it was also possible to show the blocking of light using this prepared sample.

TABLE 3
Results obtained with the product produced according
to the experimental conditions of Example 3.

| Solids | | 3-BZ | Zeta | | | | |
|---|---|---|---|---|---|---|---|
| Content | | Content | Potential | Viscosity | Particule Size (nm) | | |
| (%) | pH | (%) | (mV) | (cPs) | D₁₀ | D₅₀ | D₉₀ |
| 21.2 | 3.54 | 87 | −53.5 | 3.20 | 321 | 396 | 488 |

### Example 4

### Obtaining a Solar Protection Agent Containing Octyl Methoxycinnamate (OMC) Nanoencapsulated in Poly(Methyl Methacrylate)

In a shielded glass reactor with a bottom outlet with a volume of 200 mL, 80 g of a colloidal silica suspension was added, with a concentration by weight of 1%. After adjusting the temperature to 70° C., 20 g of methyl methacrylate and 2 g of OMC were added, while agitating at a rate of 150 rpm and the reaction was initiated by adding 0.2 g of potassium persulphate dissolved in 10 g of deionized water. The reaction was conducted at this temperature for 4 hours and after this period of time the polymeric dispersion obtained was described. The results obtained for this example are presented in Table 4.

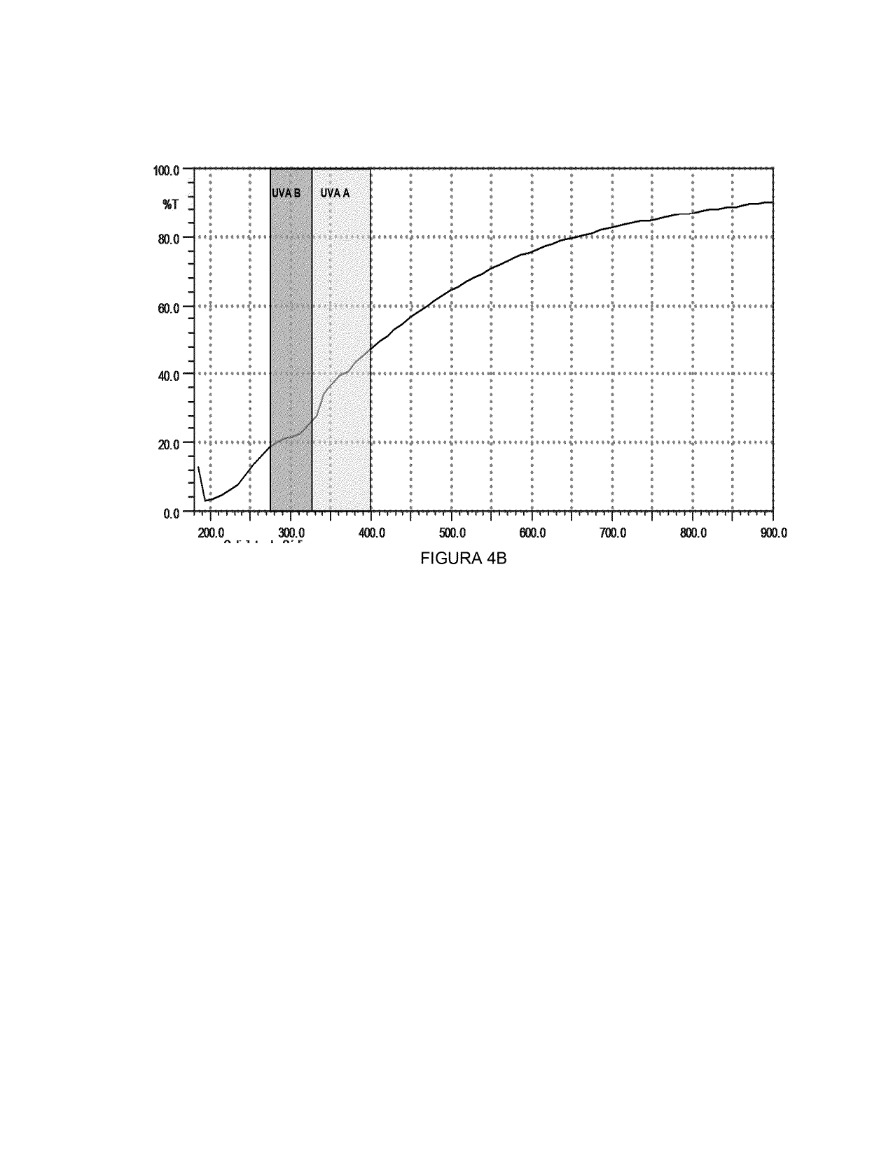

FIG. 4A presents the turbidimetric curves obtained from the polymer dispersion synthesized under the experimental conditions of this example. This result showed that for this sample of colloidal dispersion, a small phase separation occurred, indicated by a reduction in the backscattering intensity after 3 days of analysis. Nevertheless, this separation is reversible, since gentle agitation of the sample in the flask is sufficient to resuspend the particles. FIG. 4B presents the transmittance curve of a sample diluted at a concentration of 0.005% by weight. The result allows the capacity of the nanoparticles to absorb light primarily at wavelengths of less than 400 nm to be seen.

TABLE 4
Results obtained with the product produced according
to the experimental conditions of Example 4.

| Solids | | Zeta | | | | |
|---|---|---|---|---|---|---|
| Content | | Potential | Viscosity | Particule Size (nm) | | |
| (%) | pH | (mV) | (cPs) | D₁₀ | D₅₀ | D₉₀ |
| 20.95 | 5.68 | −73.9 | 3.40 | 247 | 314 | 401 |

### Example 5

### Obtaining a Solar Protection Agent Containing Octyl Methoxycinnamate (OMC) Nanoencapsulated in Poly(Methyl Methacrylate)

In a shielded glass reactor with a bottom outlet with a volume of 200 mL, 80 g of a colloidal silica suspension was added, with a concentration by weight of 1%. After adjusting the temperature to 70° C., 20 g of methyl methacrylate and 4 g of OMC were added, while agitating at a rate of 150 rpm and the reaction was initiated by adding 0.2 g of potassium persulphate dissolved in 10 g of deionized water. The reaction was conducted at this temperature for 4 hours and after this period of time the polymeric dispersion obtained was described. The results obtained for this example are presented in Table 5.

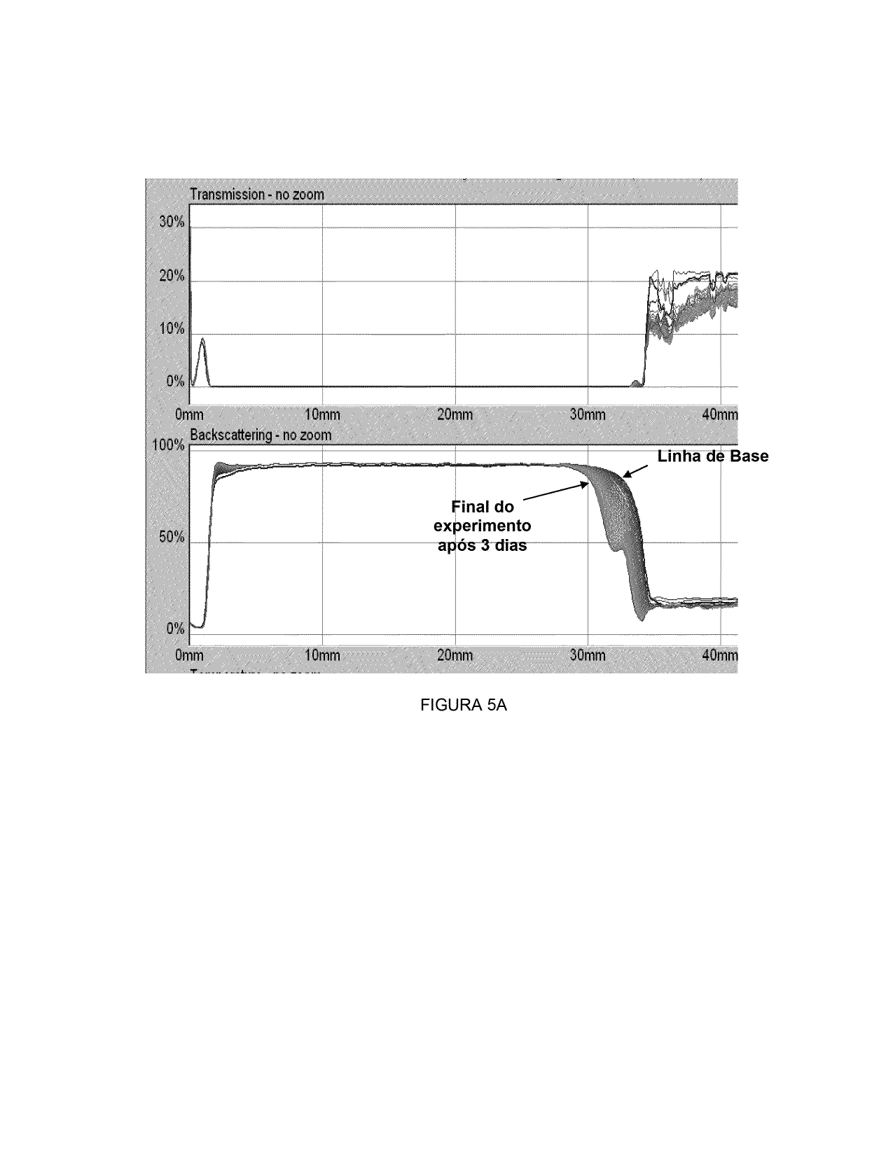

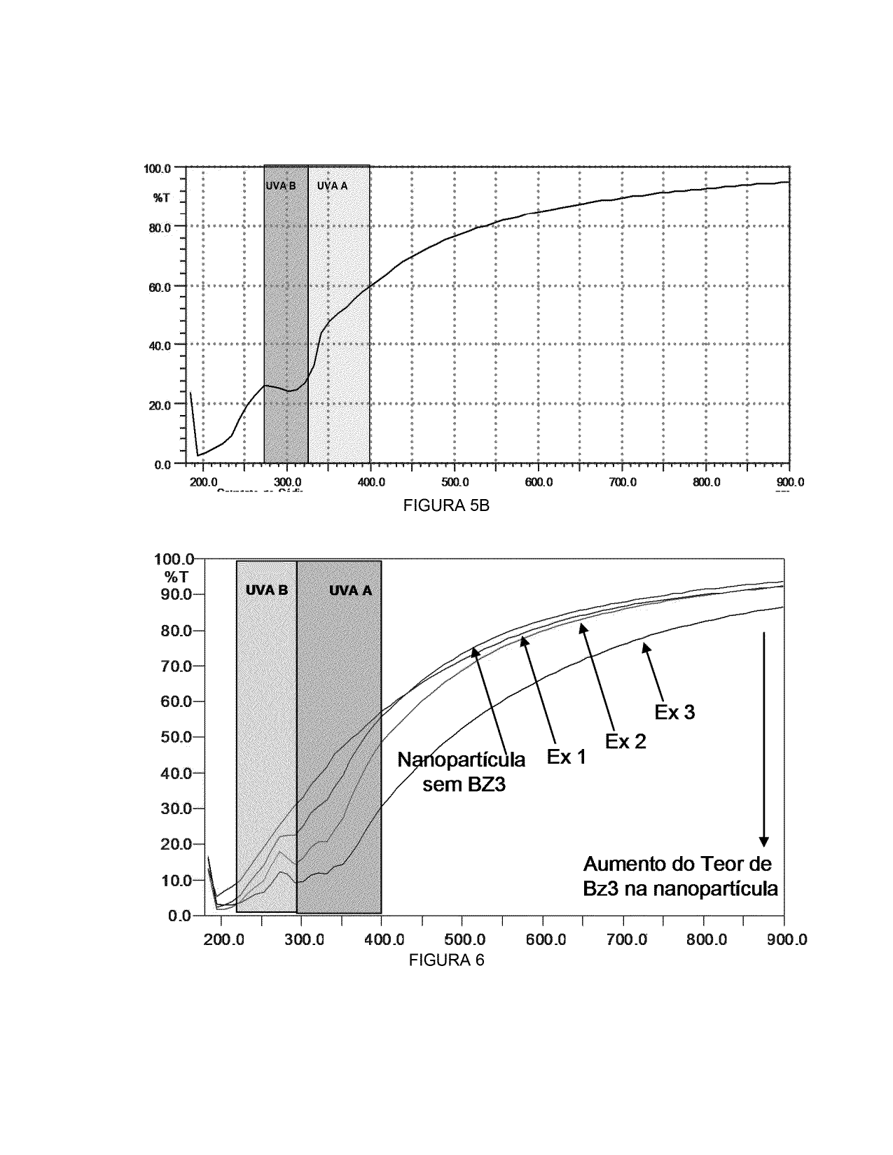

FIG. 5A presents the turbidimetric curves obtained from the polymer dispersion synthesized under the experimental conditions of this example. This result showed that for this sample of colloidal dispersion a small phase separation occurred, indicated by a reduction in the backscattering intensity after 3 days of analysis. Nevertheless, this separation is reversible, since gentle agitation of the sample in the flask is sufficient to resuspend the particles. FIG. 5B presents the transmittance curve of a sample diluted up to a concentration of 0.005% by weight. What can be seen with this result is the capacity of the nanoparticles to absorb light primarily at wavelengths of less than 400 nm.

TABLE 5
Results obtained with the product produced according
to the experimental conditions of Example 4.

| Solids | | Zeta | | | | |
|---|---|---|---|---|---|---|
| Content | | Potential | Viscosity | Particule Size (nm) | | |
| (%) | pH | (mV) | (cPs) | D₁₀ | D₅₀ | D₉₀ |
| 19.35 | 3.65 | −52.0 | 3.22 | 194 | 241 | 298 |

### Example 6

### UV Spectroscopy Transmittance Curves of the Nanostructured Solar Protection Agent Containing 3-Benzophenone

The superposition of transmittance vs. wavelength curves of samples of a protection agent synthesized in the examples according to this invention demonstrate the influence of the concentration of the chemical agent on the reduction of the passage of light by the diluted sample, indicating that the greater the concentration of the nanoencapsulated chemical agent, the greater the efficiency in blocking light will be.

### REFERENCES

Angeli V W. Desenvolvimento e Caracterização de Formulações Fotoprotetoras contendo Nanocápsulas [Development and characterization of photoprotective formulatons containing nanocapsules]. Doctoral Thesis—UFRGS, Porto Alegre, Brazil, 2007.
Flori J., Davolosi M R., Correall M A. Sunscreens. Quim Nova, v0, n1. São Paulo, 2007.
Gawkrodger D. Dermatologia [Dermatology]. Rio de Janeiro: Guanabara Koogan, 2002.
Schulz J., Hohenberge H., Pflucker F., Garter E., Will T., Pfeiffer S., Wepf R., Wendel V., Gers-Barlag H., Wittern P. Distribution of sunscreens on skin. Adv Drug Del Rev. Suppl1, v54, p157-163, 2002.
Wolf R., Will D., Morganti, Ruocco V. Sunscrens. Clinics in Dermatology, v19, 452-459; 2001.
Wright, M W., Wright, S T., Wagner, R F., Mechanisms of sunscreen failure. J Am Acad Dermatol, v44, p781-784, 2001.
GB 2453195, Aqueous nano-particulate dispersion comprising a UV absorber/polymer mixture for pharmaceutical/cosmetic applications; 2009.
US 2005/0208087. Modified Oxidic Nano-particle with Hydrophobi Inclusions, Method for the Production and Use of said Particle: 2005.
US 2009117384-A1. Metal oxide nanoparticle containing composition for UV-blocking composition used in sunscreen composition, has regular polyhedral nanoavities which are isolated from surface of nanoparticles; 2009.
U.S. Pat. No. 5,955,091. Photobluing/whitening-resistant cosmetic/dermatological compositions comprising TiO2 pigments and deformable hollow particulates; 1999.
U.S. Pat. No. 7,344,591. Stabilized titanium dioxide nanoparticle suspension, e.g. for personal care application such as sunscreen, comprises dispersing agent containing organic molecules that have functional group, e.g. hydroxyl and/or carboxyl; 2008.
WO/2003/072077. A Substantially Visibly Transparent Topical Physical Sunscreen Formulation; 2003.

1. “NANOSTRUCTURED SOLAR PROTECTION AGENT,” characterized in that it is a product with a combination of two solar protection mechanisms (physical and chemical) in the same nanoparticle that has a shell-core type of morphology, wherein the shell is formed by nano-particulates of oxides and the core is formed by a polymer, and a chemical solar radiation protection agent, the oxides being silica oxides, titanium oxide and zinc oxide, the core polymer being created from hydrophobic monomers, such as the acrylate, methacrylate, vinyl, styrene, acrylamide and methacrylamide types, and the chemical radiation protection agent being any agent according to the prior art.

2. “NANOSTRUCTURED SOLAR PROTECTION AGENT,” according to claim 1, characterized in that the core polymer is acrylates and methacrylates.

3. “NANOSTRUCTURED SOLAR PROTECTION AGENT,” according to claim 1, characterized in that the product particles are less than 1000 nm and the granulometry of the oxides is compatible with the desired granulometry of the final product.

4. “NANOSTRUCTURED SOLAR PROTECTION AGENT,” according to claim 1, characterized in that the weight ratio of oxides is 1%, that of monomers is 20% and that of protection agents is 4%.

6. “PROCESS” for production of a nanostructured solar protection agent, according to claim 1, characterized in that the synthesis to be conducted in a conventional polymerization reaction consists of a colloidal oxide dispersion prepared directly in the reactor and heated at the reaction temperature; then, the monomer charge is added, together with the chemical solar protection agent previously solubilized in the monomers, slightly homogenizing the reaction medium and adding the initiator to start the polymerization reaction, and at the end of the reaction the polymer dispersion can be used as is, without any post-processing stage.

7. “PROCESS” for production of a nanostructured solar protection agent, according to claim 6, characterized by the polymeric dispersion being recovered in the form of a powder by conventional particulate drying methods.

8. “PROCESS” for production of a nanostructured solar protection agent, according to claim 6, characterized by the colloidal dispersion of the oxide being heated to the reaction temperature, which varies based on the hydrophobic monomer used to form the polymer.

9. “PROCESS” for production of a nanostructured solar protection agent, according to claim 6, characterized by the agitation for homogenization of the reaction medium after the addition of monomers being at 10 to 1000 rpm.

10. “PROCESS” for production of a nanostructured solar protection agent, according to claim 6, characterized by the agitation for homogenization of the reaction medium after the addition of monomers being 200 rpm.

11. “PROCESS” for production of a nanostructured solar protection agent, according to claim 6, characterized by the polymerization reaction being conducted for varying lengths of time, which may range from 1 up to 24 hours of reaction, and at temperatures ranging from 50 to 100 ° C., depending on the materials used.
